(19) United States
(12) Patent Application Publication
(10) Pub. No.: US 2020/0308524 A1
(43) Pub. Date: Oct. 1, 2020

(54) Device and Method for Enhanced Air Bubble Removal from Rotary Bioreactor Cell Cultures and Culture Chambers

(71) Applicant: Temple University-of The Commonwealth SAystem of Higher Education, Philadelphia, PA (US)

(72) Inventors: Michael A. Phelan, Philadelphia, PA (US); Peter I. Lelkes, Cherry Hill, NJ (US)

(21) Appl. No.: 16/755,337

(22) PCT Filed: Oct. 9, 2018
(86) PCT No.: PCT/US18/54937

§ 371 Date: Apr. 10, 2020

Related U.S. Application Data
(60) Provisional application No. 62/570,177, filed on Oct. 10, 2017.

(51) Int. Cl. C12M 1/00; C12M 3/04

(52) U.S. Cl. CPC C12M 29/20; C12M 27/10

(57) ABSTRACT

A culture chamber for a rotary bioreactor includes a central chamber and an elongate channel having a channel entrance configured to receive bubbles from the central chamber. The elongate channel surrounds a majority of the central chamber. A method for removing bubbles from a cell culture is also disclosed.

### CROSS-REFERENCE TO RELATED APPLICATIONS

This application is a national stage application of PCT Application No. PCT/US18/54937, filed Oct. 9, 2018, and claims priority to U.S. provisional application No. 62/570,177 filed on Oct. 10, 2017, both of which are incorporated herein by reference in their entireties.

### BACKGROUND OF THE INVENTION

High aspect ratio vessels (HARV), also referred to as slow-turning lateral vessels (STLV), rotating wall vessels (RWV) and clinostat bioreactors are specific types of a rotary bioreactor that provide conditions of “simulated microgravity” (SMG) which causes cells to form aggregates, express genes differently, and induce a host of other scientifically relevant behaviors. SMG has a broad array of applications from space biology studies, to developmental biology, to tissue engineering. To generate SMG conditions, a bioreactor is filled completely with fluid and rotated such that the motion of the fluid with the cells averages out the effects of gravity. This effect is only accomplished if the chamber is entirely filled with fluid thereby effectively creating solid body rotation. Any air bubbles present can induce a “slosh dynamic” effect that introduces turbulence and harms ideal fluid dynamics. All HARVs and STLVs possess a gas permeable membrane to allow cellular respiration and to normalize pH with incubator CO₂ levels. This membrane can allow for the passage of water vapor if the humidity level is not extremely high. Loss of liquid will rapidly create bubbles, sometimes within in a matter of hours. This demands constant monitoring and checking of the bioreactor and the incubator, and the formation of bubbles can destroy experiments that may take days or weeks to prepare.

Organoids and Spheroids in Rotating Wall Vessel (RWV) Bioreactors:

2D cell culture was the gold standard of in vitro biology for nearly a century and has been used in virtually every major discovery in cell biology. However, increasing understanding of the complexity of biological systems has led to heightened awareness of the importance of model systems that more closely replicate the true 3D environment of living tissues (Antoni, Burckel, Josset, & Noel, 2015). While extensive progress has been made in engineered 3D scaffolds, organ recellularization, and bioprinting, recent studies demonstrate the unique potential of self-assembled spheroids and organoids for discoveries in disease modeling, drug discovery, and a host of other biomedical applications (Xinaris, Brizi, & Remuzzi, 2015).

Although several protocols exist for the generation of spheroids and organoids, such as spontaneous self-assembly in non-adherent cell culture plate (Fang & Eglen, 2017) or the hanging drop method (Eder & Eder, 2017), culturing cells in the RWV bioreactor with its improved nutrient exchange yields consistent formation of organoids with superior morphology and unique, organotypic gene expression (Botta, Manley, Miller, & Lelkes, 2006; Gerecht-Nir, Cohen, & Itskovitz-Eldor, 2004; Lelkes et al., 1998; Mattei, Alshawaf, D 'abaco, Nayagam, & Dottori, 2018; Radtke & Herbst-Kralovetz, 2012; Redden & Doolin, 2011; Redden, Iyer, Brodeur, & Doolin, 2014). Recently, DiStefano, et al. demonstrated that a continuous perfusion variant of the RWV produced murine retinal organoids with properly organized photoreceptor cells significantly faster and larger than static cultures (DiStefano et al., 2018). Faster generation of larger high-fidelity organoids in the RWVs may facilitate the dissemination of these organoids for high-throughput tissue analysis in drug discovery and personalized medicine.

Bubble Formation in the Rotating Wall Vessel Bioreactor and Its Impact:

Initially developed by NASA in the 1980s, RWVs were originally intended to safely shuttle cells into space. However, it was quickly discovered that the RWV was also capable of supporting the formation of complex, 3D, tissue-like structures of relevance to biomedical research on the ground (Schwarz, Goodwin, & Wolf, 1992).

Because the low-shear, microgravity effect of the RWV relies on a circular, solid-body fluid path along its interior perimeter, the RWV requires a “zero headspace” condition, literally a continuous interface between the fluid and the wall with no air gap (Hammond & Hammond, 2001; Wolf & Schwarz, 1992). When this condition is violated due to the presence of a bubble, this circular path is interrupted, as buoyancy continually forces the bubble to the highest point of the system opposite the flow of the fluid. The presence of a bubble at this locale interrupts the fluid path, adding turbulence and fluid shear. Bubbles are easily formed because each RWV bioreactor has a gas exchange system for cellular respiration and to pH-balance the cell culture media with incubator CO2. When the incubator environment has insufficiently high humidity (below 100%), negative pressure can form as water vapor diffuses away from the bioreactor, inevitably causing dissolved gases to precipitate out, forming unwanted bubbles. The need to remove bubbles from the RWV system prior to use is widely discussed in literature (Hammond, Allen, & Birdsall, 2016; Hammond & Hammond, 2001; King & Miller, 2007; Pollack, Meaney, Levine, Litt, & Johnston, 2000; Radtke & Herbst-Kralovetz, 2012; Salerno-Goncalves, Fasano, & Sztein, 2016; Varley, Markaki, & Brooks, 2017; Wuest, Richard, Kopp, Grimm, & Egli, 2015). However, what is not widely discussed is the significance of failing to do so or an analysis of the change in shear due to the presence of a bubble.

Though the importance of removing initial bubbles is widely reported, a rational and consistent way of preventing bubble formation is not. While bubbles can enter the RWV system through leaking or improper clearing, the most difficult source of bubbles to prevent is due to gas exchange. Some authors suggest a sufficiently high humidity incubator (several water pans used simultaneously) will prevent bubble formation (Hammond & Hammond, 2001). However, with the need to open the incubator for media changes or removal of samples at different time points, this becomes difficult to maintain in practice, resulting in the frequent formation of small bubbles within 24-48 hours, which in turn can act as nucleation points for large bubble growth. Over longer time points, such as that required for organoid differentiation, bubble prevention becomes virtually unsustainable. With the potential for the proliferation of RWV bioreactor technology for organoid research, there is a need for an improved system capable of neutralizing the impact of incidental bubbles.

Accordingly, there is a need in the art for an improved HARV or STLV rotary bioreactor device that has means for instantly and continuously removing bubbles from the bioreactor without disruption to fluid dynamics, all while minimizing the maintenance and disruption to the operation of the rotary bioreactor. Embodiments of the invention described herein meet these needs.

### SUMMARY OF THE INVENTION

In one embodiment, a culture chamber for a rotary bioreactor includes a central chamber; an elongate channel comprising a channel entrance configured to receive bubbles from the central chamber; wherein the elongate channel surrounds a majority of the central chamber. In one embodiment, the channel entrance comprises a back wall that is common to an outer wall to the elongate channel. In one embodiment, the elongate channel terminates in a channel exit port. In one embodiment, the elongate channel is curved. In one embodiment, the curvature of the elongate channel approximates a curvature of the perimeter of the central chamber. In one embodiment, the elongate channel surrounds the entire central chamber. In one embodiment, the elongate channel extends beyond one complete turn around the central chamber. In one embodiment, the channel entrance comprises a gating mechanism. In one embodiment, the gating mechanism is one of a hydrophobic membrane, a ball valve or a one-way flow valve. In one embodiment, the culture chamber is substantially disc-shaped. In one embodiment, the culture chamber is substantially drum-shaped. In one embodiment, the channel entrance is disposed at the vertex of at least one angled ramp. In one embodiment, the channel entrance is disposed between and at the vertex of at least two angled ramps. In one embodiment, the culture chamber includes an outer sheath at least partially comprising the elongate channel. In one embodiment, a culture chamber assembly includes a plurality of disks that form the culture chamber. In one embodiment, the culture chamber assembly includes a gas-permeable membrane layer. In one embodiment, the culture chamber assembly includes a gasket layer at least partially comprising one of the central chamber and the elongate channel. In one embodiment, a bioreactor system includes the culture chamber and a rotator device configured to attach to and rotate the culture chamber. In one embodiment, a HARV or STLV simulated microgravity kit includes the culture chamber. In one embodiment, the channel entrance shares the same plane as the central chamber. In one embodiment, the channel entrance and the central chamber are configured in different planes. In one embodiment, the culture chamber includes a second elongate channel comprising a second channel entrance configured to receive bubbles from the central chamber. In one embodiment, the channel entrance, the second channel entrance and the central chamber are all configured in different planes. In one embodiment, the central chamber is configured to accept a working volume of 10 mL of media or less.

In one embodiment, a method for removing bubbles from a cell culture includes the steps of introducing media into a culture chamber, continuously rotating the culture chamber, affecting the movement of bubbles from a central chamber of the culture chamber to a peripheral channel of the culture chamber as the culture chamber rotates, and maintaining the bubbles in the peripheral channel as the culture chamber rotates. In one embodiment, the step of introducing media into a culture chamber includes introducing media of 10 mL or less.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is side view of a culture chamber according to one embodiment;

FIGS. 2A-2D are side views of the culture chamber shown in FIG. 1, depicting bubble position at various rotational positions of the culture chamber according to one embodiment;

FIG. 3A is a face panel disk of a culture chamber assembly according to one embodiment, FIG. 3B is a channel gasket of a culture chamber assembly according to one embodiment, FIG. 3C is a channel disk of a culture chamber assembly according to one embodiment, FIG. 3D is a gas-permeable membrane of a culture chamber assembly according to one embodiment, FIG. 3E is a back panel disk of a culture chamber assembly according to one embodiment, and FIG. 3F is an exploded view of a culture chamber assembly according to an alternate embodiment;

FIG. 4A is a side view and FIG. 4B is an alternate side view of a culture chamber drum according to one embodiment, and FIG. 4C is a side view of a culture chamber drum sheath according to one embodiment; and

FIG. 5 is a flow chart of a method for removing bubbles from a cell culture according to one embodiment.

FIG. 6A is a perspective view of a Discontinuous Perfusion Bioreactor (DPB) according to one embodiment, and FIG. 6B is a diagram of a prior art Continuous Perfusion Bioreactor (CPB).

FIGS. 7A-7H show side and perspective views of fabrication and assembly of prior art (FIGS. 7A and 7C-7E) and novel (FIGS. 7B and 7F-7H) RWV bioreactors according to one embodiment. Disassembled bioreactor parts (FIGS. 7C, 7F), completely assembled devices (FIGS. 7D, 7G), and bioreactors filled with colored liquid to visualize the internal volume (FIGS. 7E, 7H). Scale bars for FIGS. 7A-7C and FIG. 7F are 5 cm; and FIGS. 7D, 7E, 7G and 7H are 2.5 cm.

FIGS. 8A-8L show side views of the design and modeling of a RWV bioreactor according to one embodiment. The figures show (FIG. 8A) the design according, (FIG. 8B) the behavior of a bubble contained to the channel, (FIG. 8C) the area modeled in the ANSYS CFD software, (FIG. 8D) a completed CFD mesh showing the size and distribution of individual voxels, (FIGS. 8E, 8F) a model of fluid flow vectors in a standard design bioreactor at 0.1 seconds and 15 minutes respectively, (FIGS. 8G, 8H) a model of fluid flow vectors in a novel design bioreactor at 0.1 seconds and 15 minutes respectively, (FIGS. 8I, 8J) a bubble entering the channel, (FIGS. 8K, 8L) a bubble moving through the channel to the back wall, (FIGS. 8I-8L) for better visualization the fluid (water) has been stained with red food color. The bubble is shown by the yellow arrows in FIGS. 8I-8L. Scale bars for FIGS. 8E-8F represent 2 cm, and FIGS. 8I-8L are 2.5 cm.

FIGS. 9A-9D show images of alginate bead shear modeling. The motion of the alginate beads are taken every 1/20th of a second in RWV bioreactors rotating counter-clockwise under the four defined conditions. The bubble is shown by the arrow in FIGS. 9C and 9D. FIG. 9E shows the average fluid shear experienced by the beads over the course of the path. The shear observed in the novel design with bubble condition is not significantly different from that in the first two instances. FIG. 9F shows the maximum shear experienced by the beads during the observed path. All scale bars are 2.5 cm.

FIGS. 10A-10N represent A549 cell culture results for spheroid morphology (FIGS. 10A-10C) observed results for the design according to one embodiment with no bubble present, (FIG. 10D-10F) observed results for a prior art design with no bubble present, (FIGS. 10G-10I) observed results for the design according to one embodiment with a bubble present, (FIGS. 10J-10L) observed results for a prior art design with a bubble present, (FIG. 10M) combined quantified results for spheroid areas for each condition taken as the average of medians, and (FIG. 10N) combined quantified results for spheroid circularity taken as an average of averages of each replicate. All scale bars represent 200 μm.

FIGS. 11A and 11B show according to one embodiment experimental analysis of failure mode (FIG. 11A) the path by which the “channel capture” failure mode may occur as solid body rotation is established, and (FIG. 11B) the incidence of that failure mode with human dermal fibroblasts over 24 hours.

### DETAILED DESCRIPTION

### Definitions

Unless defined otherwise, all technical and scientific terms used herein have the same meaning as commonly understood by one of ordinary skill in the art to which embodiments of this invention belongs. Although any methods and materials similar or equivalent to those described herein can be used in the practice or testing of embodiments of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “simulated microgravity” refers to the creation of a solid rotational body of fluid in which the gravitational vector is averaged to near zero for particles suspended in said fluid.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to novel vessels for rotary cell culture bioreactors. In some embodiments, the present invention provides novel high aspect ratio vessels (HARV) for rotary cell culture bioreactors. A HARV is a type of rotary bioreactor which can expose cells to simulated microgravity conditions. In some embodiments, the present invention provides novel slow turning lateral vessels for rotary (STLV) cell culture bioreactors. An STLV operates much like a HARV but has been extended along its axis of rotation.

Embodiments of the invention described herein actively and continuously remove gas bubbles from the system before they are able to affect bioreactor performance. A structure such as a disc housing the main volume includes a thin channel that runs around the majority of the perimeter of the disk. The channel captures bubbles that move away from the main volume during rotation, allowing the bubbles to maintain a position significantly far away from the main volume so that they do not interact with the main body of the fluid. As a result, bubbles are instantly and continually removed from the main body of fluid, there is no disruption to fluid dynamics, the system requires less observation and upkeep for experiments, experiments are less likely to fail, and incubators can run at lower humidity with fewer water trays and are less likely to experience contamination.

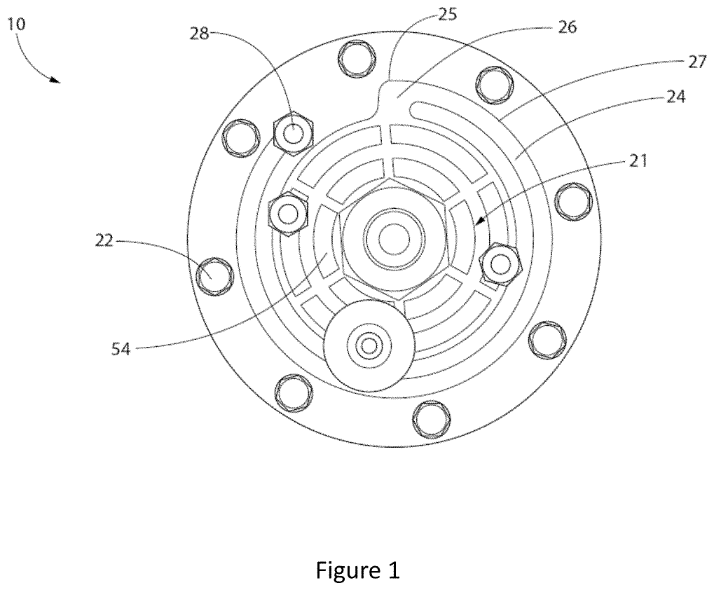

With reference now to FIG. 1, embodiments of the invention include a HARV media chamber 10 in the shape of a disc that contains the main volume of fluid within a central chamber 21. An elongate channel 24 is connected to the central chamber 21 via a channel entrance 21. The channel entrance 21 includes a back wall 25 of the elongate channel 24. The back wall 25 is spaced away from the central chamber 21 allowing bubbles to rise and collect into the channel entrance 21. The back wall 25 is also common to the outer wall 27 of the elongate channel 24, so that the collected bubbles naturally move further into the elongate channel 24 as the HARV media chamber 10 rotates, explained in more detail below. The elongate channel 24 terminates in a channel exit port 28. In certain embodiments, the disk is a multi-layer assembly that is connected by a fastener 22 such as a screw. Examples of the multi-layer assembly are provided further below. In certain embodiments, gas vents 54 are provided and oriented in an array of discontinuous concentric curves.

In one embodiment, the elongate channel is a curved channel. In one embodiment, the curvature of the channel is the same or close to the perimeter of the central chamber 21 and/or the perimeter of the HARV disk. In one embodiment, the elongate channel 24 runs around less than or about half the perimeter of the central chamber 21. In one embodiment, the elongate channel 24 runs around the majority of the perimeter of the central chamber 21. In one embodiment, the elongate channel 24 runs around the entire perimeter of the central chamber 21. In certain embodiments, the elongate channel 24 can continue to run past one complete turn around the perimeter of the central chamber 21, for instance making 1.5 or 2 or more turns around the perimeter of the central chamber 21. In one embodiment, the elongate channel includes a combination of straight sections and/or curved sections. In one embodiment, the elongate channel 24 includes a constant width. In one embodiment, the elongate channel 24 tapers up to the channel exit port 28. In one embodiment, the elongate channel 24 tapers down to the channel exit port 28. In one embodiment, the elongate channel 24 has a variable width between the channel entrance 21 and the channel exit port 28.

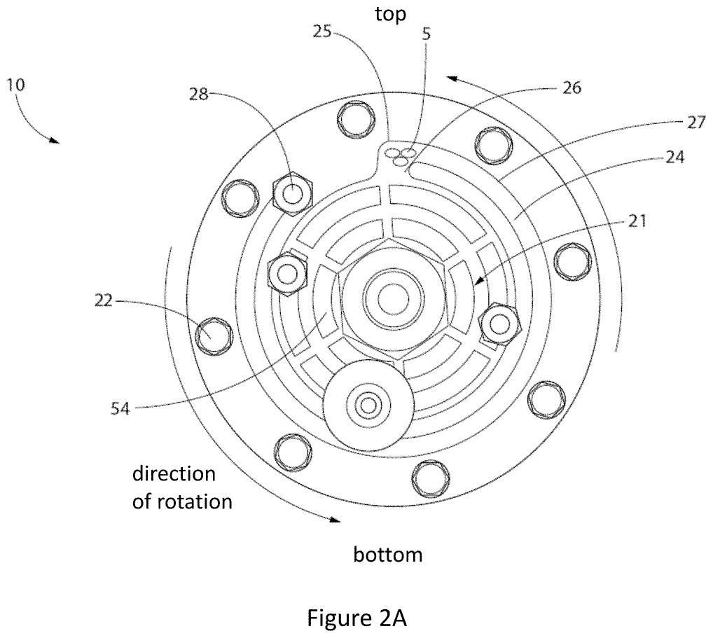

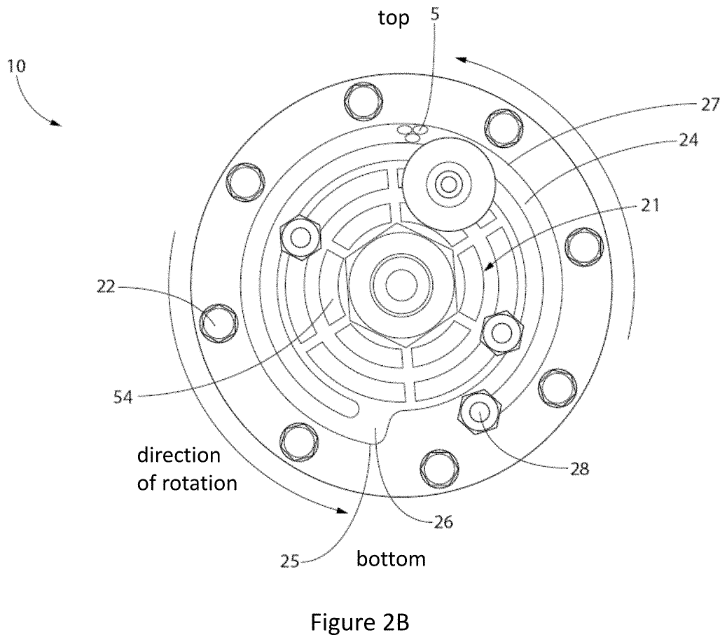

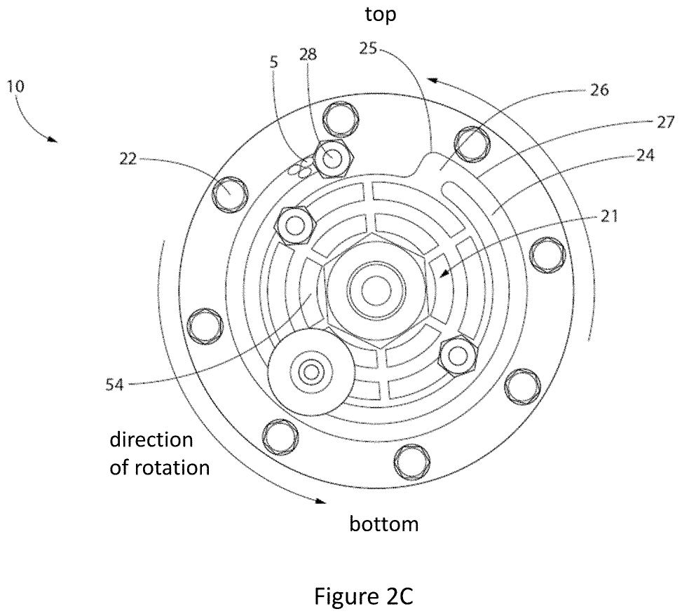

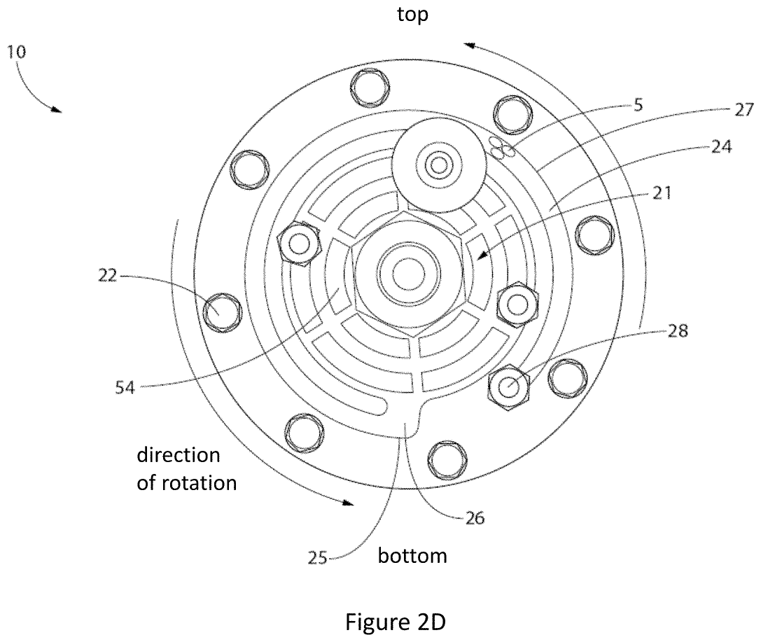

With reference now to FIGS. 2A-2D, the elongate channel 24 runs around the majority of the perimeter of the central chamber 21. As the HARV media chamber 10 rotates (counter-clockwise in this example), any bubbles rise to the top due to buoyancy. When the channel entrance 26 is at a top position of the HARV media chamber 10 (e.g. FIG. 2A), the bubbles 5 enter the elongate channel 24 as they are displaced by media that was added to the channel before the experiment began. When the bubbles 5 encounter the back wall 25 of the elongate channel 24, they are pulled around the circumference of the channel approximately until the wall reaches the “3 o'clock” position and the bubbles float upwards. As the HARV media chamber 10 rotates further (e.g. FIG. 2B), the bubbles 5 stay on the top, moving further and further away from the channel entrance 26 and along the elongate channel 24 as the channel entrance 26 approaches a bottom position of the HARV media chamber 10. Because the elongate channel 24 is sufficiently long, the bubbles 5 remain significantly distant from the channel entrance 26 and do not interact with the main body of the fluid in the central chamber 21 (e.g. see further rotation depicted in FIGS. 2C and 2D). This process can repeat itself many times. A channel exit port 28 at the back of the elongate channel 24 enables for the periodic addition of media and removal of bubbles 5 as necessary, enabling the device to be maintained indefinitely with only minor upkeep.

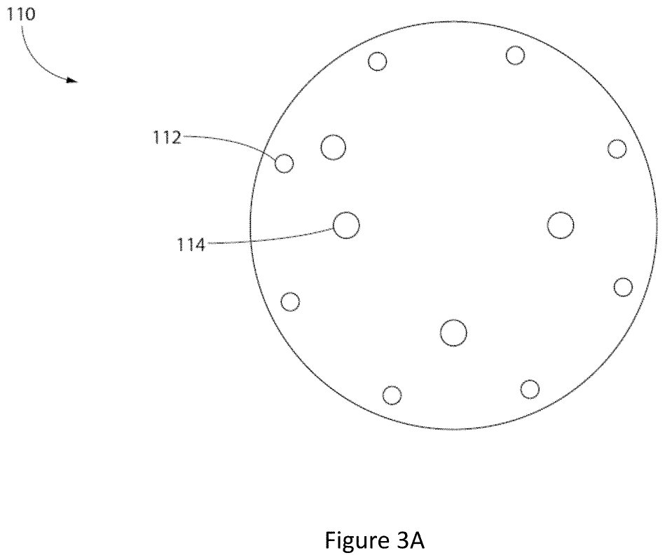

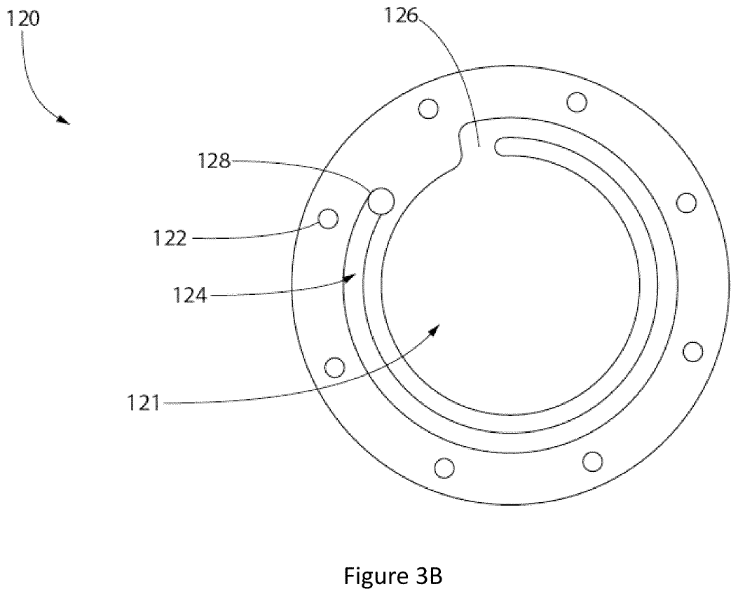

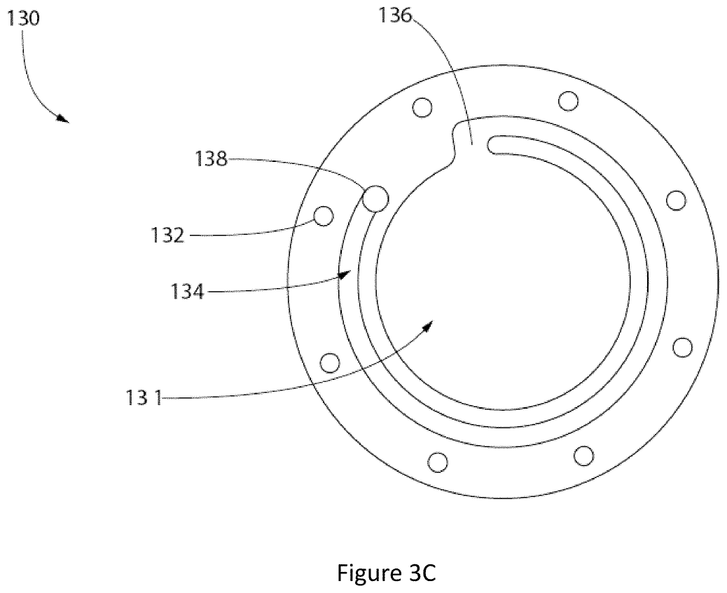

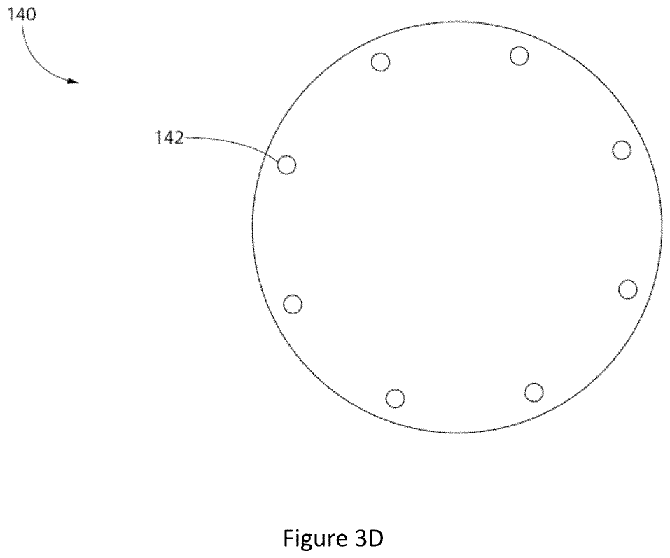

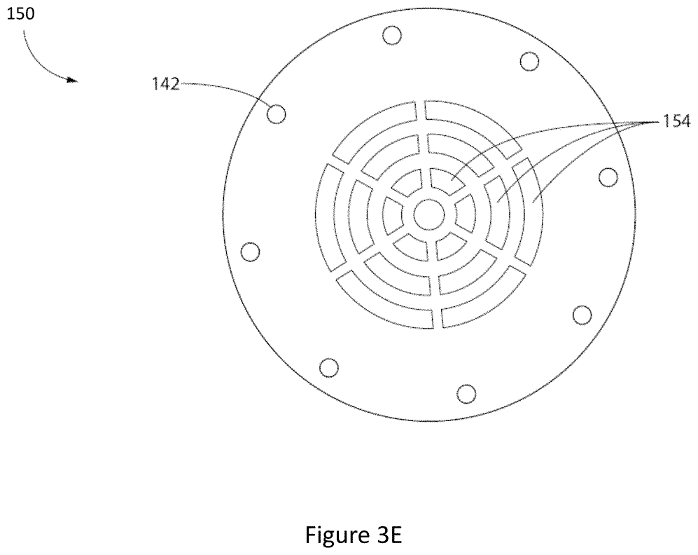

Referring now to FIGS. 3A-3E, according to one embodiment, multiple specialized disks are stacked to form a HARV assembly 100. In one embodiment, the HARV assembly 100 includes a face panel disk 110 (FIG. 3A), a channel gasket 120 (FIG. 3B), a channel disk 130 (FIG. 3C), a gas-permeable membrane 140 (FIG. 3D), and a back panel disk 150 (FIG. 3E) stacked top-down in that order. With reference to FIG. 3A, the face panel disk 110 of includes multiple assembly holes 112 and port holes 114. Assembly holes 110 may be distributed around the perimeter of face panel disk 110 and in some embodiments, are suitable for receiving assembly couplers, for example screws, pegs, rods, nails, dowels, and the like. Port holes 114 may be suitable for interfacing with fittings, for receiving fluids and/or regulating the passage of fluids, including suitable materials for the operation and maintenance of the HARV device 100, such as culture media, gases, water, buffered saline solutions, and the like. In some embodiments, port holes 114 are threaded. In some embodiments, port holes 114 are connected to one or more screw to Luer lock adapters, stopcocks, caps, one-way valves, pipe fittings, compression tube fittings, flare fittings, slip fittings, filters and the like. The face panel disk 110 can be constructed from any suitable sterilizeable material known in the art, such as acrylic, polycarbonate, polypropylene, polyethylene, stainless steel, titanium, aluminum, aluminum alloys, and the like. In some embodiments, face panel disk 110 has a diameter of about 10 mm to about 200 mm, about 25 mm to about 150 mm, about 50 mm to about 100 mm, and the like. In one embodiment, face panel disk 110 may have a diameter of about 87 mm.

Referring now to FIG. 3B, channel gasket 120 is a disk-shaped gasket comprising a central chamber 121, elongate curved channel 124 with channel entrance 126 and channel exit 128. Channel gasket 120 also includes a plurality of assembly holes 122 distributed around the perimeter of channel gasket 120, and are suitable for receiving assembly couplers such as screws, pegs, rods, nails, dowels, and the like. In some embodiments, curved channel 124 extends circumferentially around nearly the entire perimeter of channel gasket 120 forming a channel connecting to central chamber 121 at channel entrance 126 and extending substantially parallel to and concentric with central chamber 121. In some embodiments, channel exit 128 aligns with at least one of port holes 114 of face panel disk 110. Channel gasket 120 may be constructed from any suitable sterilizable material known in the art for forming a sealing gasket, including but not limited to, for example, silicone, polytetrafluorethylene, rubber, and the like. In some embodiments, channel gasket 120 has a thickness of about 0.1 mm to about 1.0 mm. In some embodiments, channel gasket 120 has a thickness of about 0.5 mm.

Referring now to FIG. 3C, channel disk 130, comprises central chamber 121, elongate curved channel 134 with channel entrance 136 and channel exit 138. Channel disk 130 also includes a plurality of assembly holes 132 distributed around the perimeter of channel disk 130, and are suitable for receiving assembly couplers such as screws, pegs, rods, nails, dowels, and the like. In some embodiments, curved channel 134 extends circumferentially around nearly the entire perimeter of channel gasket 130 forming a channel connecting to central chamber 131 at channel entrance 136 and extending substantially parallel to and concentric with central chamber 131. In some embodiments, central chamber 131 substantially forms the primary culture chamber of HARV device 100. In some embodiments, channel disk 130 comprises a gating mechanism that may regulate flow of fluid through channel entrance 136. For example, channel entrance 136 may include a hydrophobic membrane, ball valve, one-way flow valve, and/or other suitable gating mechanism forming a selective or nonselective barrier between central chamber 131 and curved channel 134 in or around channel entrance 136. In some embodiments, channel disk 130 comprises a gating mechanism that may regulate flow of fluid through channel exit 138, for example, a flow valve such as a one-way valve, ball valve, or the like. In some embodiments, channel exit 138 aligns with channel exit 128 of channel gasket 120, and at least one of port holes 114 of face panel disk 110. In some embodiments, central chamber 131 of channel disk 130 substantially aligns with central chamber 121 of chamber gasket 120. In some embodiments, curved channel 134 substantially aligns with curved channel 124 of channel gasket 120. Channel disk 130 may be constructed from any suitable sterilizable material known in the art, for example acrylic, polycarbonate, polypropylene, polyethylene, and the like. In some embodiments, channel disk 130 has a thickness of about 1 mm to about 20 mm, about 2 mm to about 10 mm, and the like. In one embodiment, channel disk 130 may have a thickness of about 4.5 mm. In some embodiments, channel disk 130 has an interior diameter of about 10 mm to about 100 mm, about 25 mm to about 75 mm, and the like. In one embodiment, channel disk 130 has an interior diameter of about 51 mm. In some embodiments, channel disk 130 has an exterior diameter of about 10 mm to about 150 mm, about 20 mm to about 120 mm, about 50 mm to about 100 mm, and the like. In one embodiment, channel disk 130 has an exterior diameter of about 87 mm.

Referring now to FIG. 3D, gas-permeable membrane 140 is a disk-shaped membrane comprising a plurality of assembly holes 142 distributed around the perimeter of membrane 140, and are suitable for receiving assembly couplers such as screws, pegs, rods, nails, dowels, and the like. Gas-permeable membrane 140 may be constructed from any suitable gas-permeable material known in the art, for example polydimethylsiloxane, polytetrafluorethylene, nylon, polyethersulfone, and the like.

Referring now to FIG. 3E, back panel disk 150 comprises a plurality of assembly holes 152 distributed around the perimeter of back panel disk 150, and a plurality of gas vents 154. In some embodiments, assembly holes suitable for receiving assembly couplers such as screws, pegs, rods, nails, dowels, and the like. In some embodiments, the plurality of gas vent 154 are oriented in an array of discontinuous concentric curves, matrix of small circles, or continuous, multi-ringed spiral. Back panel disk 150 may be constructed from any suitable, sterilizable material known in the art, for example, acrylic, polycarbonate, polypropylene, polyethylene, stainless steel, titanium, aluminum, aluminum alloys, and the like. In some embodiments, back panel disk 150 further comprises an interlocking mechanism for attaching the assembled HARV device 100 to a rotating device or component thereof. In some embodiments, back panel disk 150 has a diameter of about 10 mm to about 150 mm, about 20 mm to about 120 mm, about 50 mm to about 100 mm, and the like. In one embodiment, back panel disk has a diameter of about 87 mm.

In some embodiments, the assembled HARV device 100 of the present invention comprises face panel disk 110, channel gasket 120, channel disk 130, gas-permeable membrane 140, and back panel disk 150. In some embodiments, the assembled HARV device 100 of the present invention comprises face panel disk 110, channel gasket 120, channel disk 130, a second channel gasket 120, gas-permeable membrane 140, and back panel disk 150. HARV device 100 is assembled by aligning assembly holes 112, 122, 132, 142, and 152 and in some embodiments, introducing a plurality of receiving assembly couplers such as screws, pegs, rods, nails, dowels, and the like through each of the assembly holes of device 100 such that a single assembly coupler extends through each of the aligned assembly holes of each component as described herein thereby sandwiching each layer of device 100 together. In some embodiments, each component of HARV device 100 comprises the same number of assembly holes such that all of the plurality of assembly holes is aligned and coupled when device 100 is assembled.

In some embodiments, the assembled HARV device of the present invention comprises an aligned first channel gasket 120, channel disk 130, and a second channel gasket 120 sandwiched between a face disk 110 on one end, and a gas permeable membrane 140 and back panel disk 150 on the other end. The central chambers of the first channel gasket 120, channel disk 130, and second channel gasket 120 are aligned thereby forming a combined central chamber comprising significantly cylindrical conformation having a high aspect ratio. Similarly, the curved channels of the first channel gasket 120, channel disk 130, and second channel gasket 120 are aligned thereby forming a combined curved channel through which air bubbles may pass. The channel entrance of the first channel gasket 120, channel disk 130, and second channel gasket 120 are significantly aligned thereby forming an entrance for bubbles to pass from the combined central chamber into the combined curved channel. The channel exit of the first channel gasket 120, channel disk 130, and second channel gasket 120 are significantly aligned thereby forming an exit through which bubbles may be expunged. In one embodiment, with reference now to FIG. 3F, two or more channels 182, 184 are offset from the plane of rotation of the main volume 180. This allows the opportunity to increase the device to an arbitrarily high number of channels. Accordingly, embodiments of the device can include a channel is in-line or offset with the plane of rotation of the main chamber. Embodiments of the device can also include one or more channel entrances that can be used simultaneously.

Embodiments of the present invention further include a drum-shaped STLV device for culturing cells under simulated microgravity conditions while providing a means for eliminating bubbles that can form during normal operation such devices.

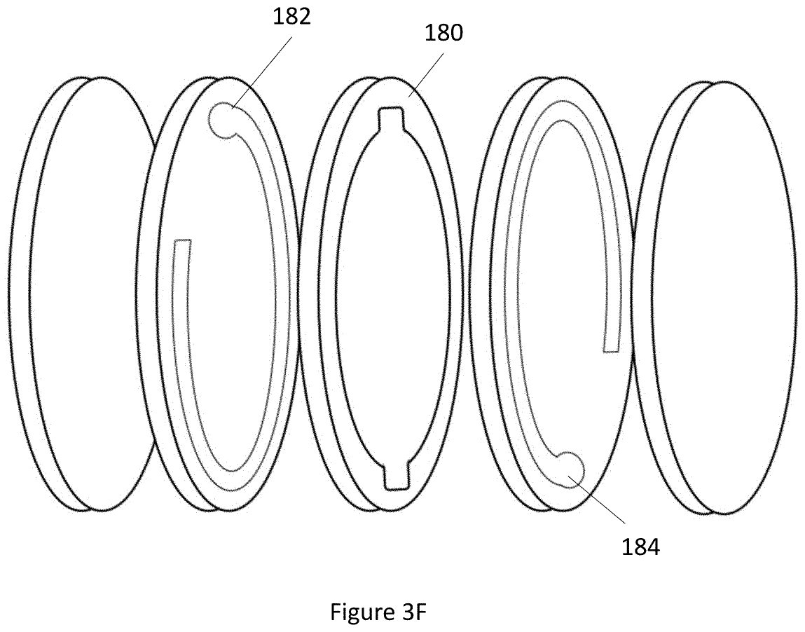

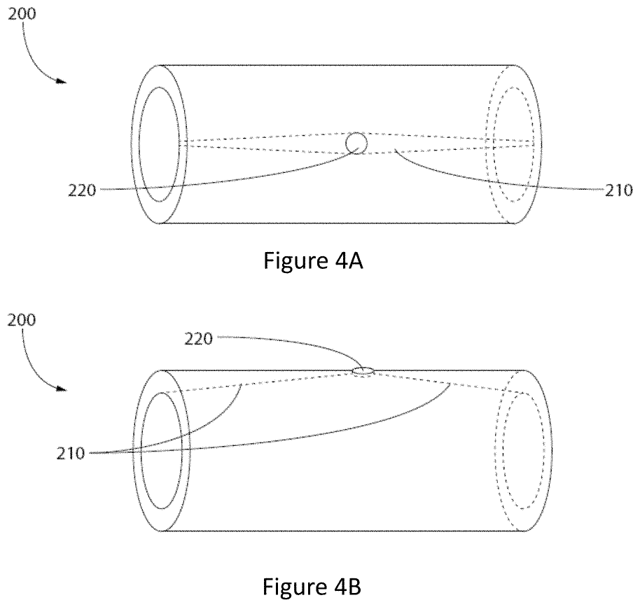

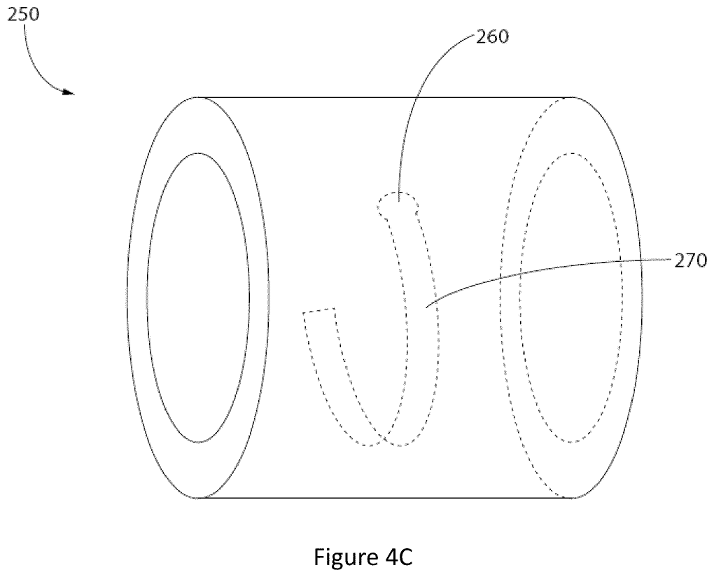

Referring now to FIGS. 4A-4C, the STLV media chamber 200 according to one embodiment comprises a significantly cylindrical vessel comprising and inner wall and an outer wall. The inner wall of STLV device 200 comprises a plurality of adjacent angled buoyancy ramps 210 extending from front wall 201 and back wall 202 meeting at bubble port 220 situated in the middle of the length of the wall of the cylindrical vessel. In some embodiments, adjacent angled ramps 210 extend along a single length of the inner wall of device 200 thereby forming a single vertex. In some embodiments, adjacent angled ramps 210 span the entire perimeter of the inner wall of device 200 thereby forming a continuous radial vertex. In some embodiments, the front wall 201 and back wall 202 of device 200 may comprises one or more ports for introducing fluid, cells, and other suitable materials for culturing cells and/or tissues, as understood in the art. In some embodiments, bubble port 220 may be threaded or have other suitable surfaces for interfacing with a fitting thereby allowing for the removal of gas while preventing the loss of liquid from the inner chamber of device 200. In some embodiments, bubble port 220 comprises a gating mechanism that may regulate the flow of fluid through bubble port 220, for example, a gas-purging valve, a gas-permeable membrane and/or other suitable gating means as understood in the art. In some embodiments, STLV device 200 further comprises sheath 250, shown in FIG. 4C. Sheath 250 comprises inlet port 260 and channel 270 and may be placed over STLV device 200 such that inlet port 260 is aligned with the bubble port 220. In some embodiments, sheath 250 comprises a gating mechanism that may regulate flow of fluid through inlet port 260 such that gas bubbles may pass through inlet port 260 but liquids may not pass. For example, inlet port 260 may include a hydrophobic membrane, ball valve, one-way flow valve, and/or other suitable gating mechanism forming a selective or nonselective barrier between bubble port 220 and channel 270 in or around inlet port 260. The STLV device 200 may be constructed from any suitable sterilizable material known in the art, for example acrylic, polycarbonate, polypropylene, polyethylene, stainless steel, titanium, aluminum, aluminum alloys, and the like.

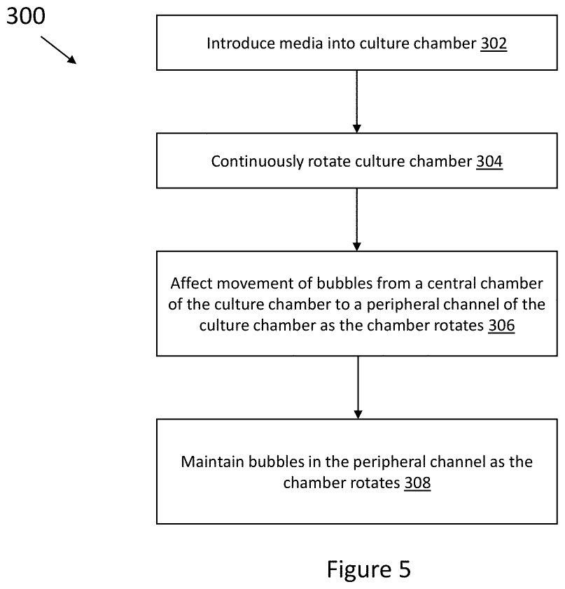

Embodiments of the present invention also provides methods for culturing cells under simulated microgravity conditions using the various embodiments of the HARV device and/or the STLV device as described herein. With reference now to FIG. 5, in one embodiment, a method 300 for separating bubbles from a cell culture includes the steps of introducing media into a culture chamber 302, continuously rotating the culture chamber 304, affecting the movement of bubbles from a central chamber of the culture chamber to a peripheral channel of the culture chamber as the culture chamber rotates 306, and maintaining the bubbles in the peripheral channel as the culture chamber rotates 308. Affecting the movement of bubbles can simply be based on continued rotation and principals of chamber geometry and structure disclosed in the embodiments herein. In certain embodiments, rotation is a pulsatile or intermittent rotational movement of the device instead of a smooth continuous rotation. In some embodiments, cells and culture media are introduced into the central chamber of the device by way of one or more of the ports holes of one or more embodiments of the devices as described herein. In some embodiments, cells are cultured under suitable conditions as understood by those skilled in the in the art such that the cells adhere to one or more surfaces of the central chamber of the device. Alternatively, nonadherent cells are introduced into the central chamber of the device by way of one or more of the port holes as described herein. The fluid level within the vessel maintained such that the vessel is fluid-tight and the device is mounted onto an appropriate rotator device such that the cells are cultured under continuous rotation thereby simulating microgravity conditions. The cells are cultured under appropriate conditions such as with appropriate media exchanges and under appropriate humidity, temperature, and CO₂ conditions as understood in the art. On occasion, air bubbles may form during the normal operation of the HARVs and/or STLVs such as those described herein for various reasons including but not limited to fluid loss due to changes in the ambient humidity, among others. In the event an air bubble forms within the central chamber of one or more of the devices described herein, buoyant forces will direct the formed bubbles to the top of the chamber, and the bubbles will be expunged from the central chamber through one or more of the perimeter channels as described herein thereby reducing the risk of disruption of fluid mechanics within the vessel and reducing the risk of injuring cells within the chamber.

In one aspect, embodiments of the present invention also include a kit comprising instrumentation to apply simulated microgravity conditions to cells or tissues in culture. In certain embodiments, the kit is a sterile packaged kit. In certain embodiments, the one or more instruments of the simulated microgravity kit are sterile and contained in one or more individual sterile packages within the kit. The sterile simulated microgravity kit contemplated herein is thus immediately ready for simulated microgravity application upon removal of the instruments from their respective packages without the need for pre-operation cleaning, sterilizing, or other processing. In certain aspects, the one or more instruments of the simulated microgravity kit are single-use instruments. For example, in one embodiment, the one or more instruments of the simulated microgravity kit are sterile and disposable. In another embodiment, the one or more instruments of the simulated microgravity kit are repackaged after use, where, in certain embodiments, the one or more instruments may be reprocessed for future use. In one embodiment, the instruments may be provided in one or more blister packaging. Each blister may comprise a plastic container (e.g., PETG) component and a lid (e.g., Tyvek®) component.

In certain embodiments, the simulated microgravity kit comprises at least one HARV or STLV as described herein. In certain embodiments, the simulated microgravity kit may optionally include other instruments, such as and without limitation, one or more fittings, tubing, pumps, rotators, bioreactors, and/or reservoirs. For example, in one embodiment, the simulated microgravity kit comprises one or more fittings, which may be barbed fittings, threaded, slip, compression, flare, flange, crimped, and/or pressed fittings or the like, or a combination thereof. In one embodiment, the fluid kit comprises one or more lengths of tubing, including any size or geometry of tubing which may be necessary for the desired application. In one embodiment, the tubing is composed of Tygon®, nylon, polyethylene, or other suitable material known to one skilled in the art. In one embodiment, the tubing comprises one or more stops for integration into holders of a peristaltic pump. In one embodiment, the simulated microgravity kit comprises one or more rotators or bioreactors. In one embodiment, the one or more rotators or bioreactors are single speed, multi-speed, single station, multiple station rotators or bioreactors, or the like, as known to one skilled in the art. In one embodiment, the simulated microgravity kit may include one or more HARVs or STLVs or culturing wells. In one embodiment, the simulated microgravity kit may include culturing media or culturing media components for admixing. In one embodiment, the simulated microgravity kit may include a biological material, such as a cell or tissue.

In certain embodiments, the simulated microgravity kit is custom-configured with regard to size and geometry appropriate to the well being used. In one embodiment, the kit is configured for a specific size of cylindrical body or flange. In an alternative embodiment, the kit comprises instrumentation that may be necessary for various sizes of vessels.

In certain embodiments, the kit comprises instructional material. Instructional material may include a publication, a recording, a diagram, or any other medium of expression which can be used to communicate the usefulness of any of the HARVs or STLVs or simulated microgravity kit components described herein. The instructional material may also include description of one or more steps to perform any of the methods described herein. The instructional material of the kit may, for example, be affixed to a package which contains one or more instruments which may be necessary for the desired procedure. Alternatively, the instructional material may be shipped separately from the package, or may be available electronically, such as accessible from the Internet, or any downloadable electronic document file format.

Still further, embodiments of the present invention include a method of providing a sterile simulated microgravity kit as described herein. In certain embodiments, the method comprises receiving a request for one or more instruments for use in culturing cells or the like. In certain embodiments, the method comprises a customized request for particular instrumentation. In certain embodiments, the method comprises a request for a standardized kit which would contain the one or more instruments. In certain instances, the method comprises gathering the one or more instruments which were requested. In one embodiment, the method comprises processing the one or more instruments. For example, in one embodiment, the one or more instruments are sterilized. Sterilization of the one or more instruments may be conducted by any suitable method known in the art. In one embodiment, the method comprises packaging the one or more instruments. For example, the one or more instruments may be packaged in one or more sterile packages to form a sterile kit.

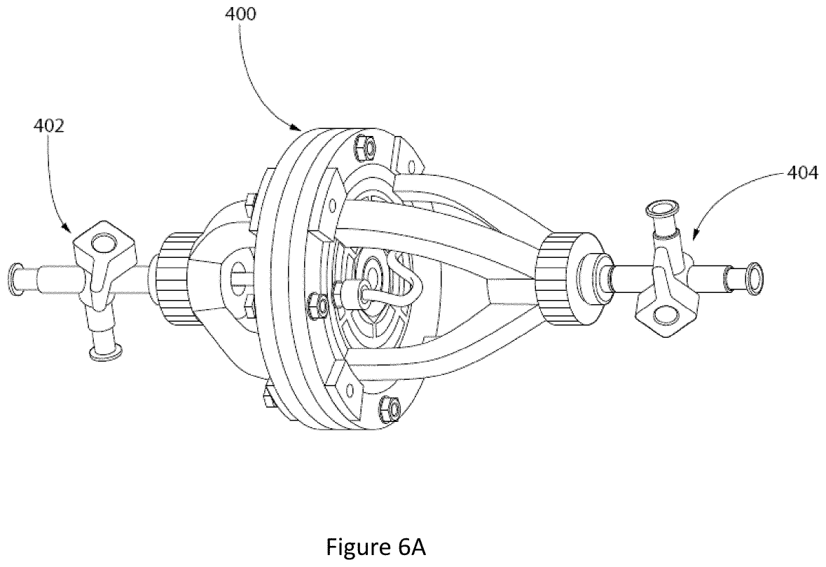

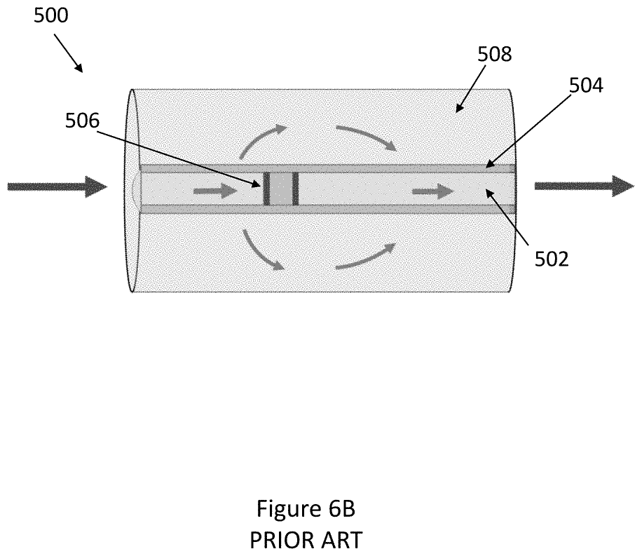

In one embodiment, the culture chamber filters bubbles, and is also configured to facilitate media changes, such as adding nutrients. In one embodiment, the advantages to the culture chamber discussed herein facilitate miniaturized bioreactor technology. With reference now to FIG. 6A, in one embodiment, a Discontinuous Perfusion Bioreactor (DPB) leverages its media change capacity of the channel with an automated pumping system and gas exchange column. The DPB includes a media chamber 400 and in-line rotary stopcocks 402, 404 for uninterrupted rotation during pumping, and provide a significant advantage over conventional “Continuous Perfusion Bioreactors” (CPB).

With conventional CPBs 500 (see e.g. FIG. 6B), to perform media changes, the CPBs have a small, hollow microporous column 502 having walls made from a microporous material 504 that runs the length of the central axis of the interior of the vessel with a small impermeable plug 506 placed a set distance near the middle. Media 508 is pumped into one side of the column 502 and withdrawn from the other side simultaneously, deflecting around the plug 506 as it's added to create an exchange “field”. Inherently, this design requires that the column 502 (and subsequently the entire bioreactor cylinder) to be several inches of length so that distinct “fill” and “empty” zones exist. Because these bioreactors also require a minimum diameter, the continuous perfusion system operates best at 50 mL or so with an additional 50 in reserve. As shown in more detail in prior art FIG. 6B, the media exchange system must be capable of removing old fluid, so ideally, the curved arrows in the diagram extend as far away from the central microporous structure as possible. The longer the tube, the farther out the arrows can theoretically extend.

Embodiments of the DPB system and device described herein replace the conventional mechanism by which change occurs, eliminating the need for a minimum cylinder length, allowing working volumes as low as 10 mL in the bioreactor with 5-10 in reserve. The embodiments take the media exchange system off the central axis such that the main cylinder can be made arbitrarily short. In one embodiment, the device incorporate gates or meshes at the entrance of the channel. In one embodiment, the system enables miniaturization to volumes at 10 mL or below. The cost of media in these systems can run in the tens or even hundreds of thousands of dollars over months of culture. Advantageously, the miniaturization component provides a significant advantage. In one embodiment, an angled rotation for the simulation of gravity conditions between 0 and 1 (i.e. partial gravity).

It should be appreciated that the devices and the simulated microgravity kits described herein may be used for a variety of simulated microgravity procedures including but not limited to culturing cells, tissue, biological samples, non-biological samples, and the like. The procedures may be performed on any cell in the human or vertebrate body, including, but not limited to, osteocytes, osteoblasts, osteoclasts, endothelial cells, epithelial cells, smooth muscle cells, mesenchymal cells, progenitor cells, and the like. It should be understood that the present disclosure is not limited to a specific cell, tissue, or simulated microgravity application.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

In this study, a modification to the traditional RWV was designed and implemented, which resulted in the effective capture and removal of bubbles. Comparison between the modified and standard bioreactors showed similarities in both fluid dynamics and in the generation of organoids under ideal, non-bubble conditions. While the formation of spheroids/organoids was impaired in conventional RWVs in the presence of a bubble, it was fully maintained in the modified design.

Materials and Methods

Bioreactor Design, Modeling, and Computational Fluid Dynamics:

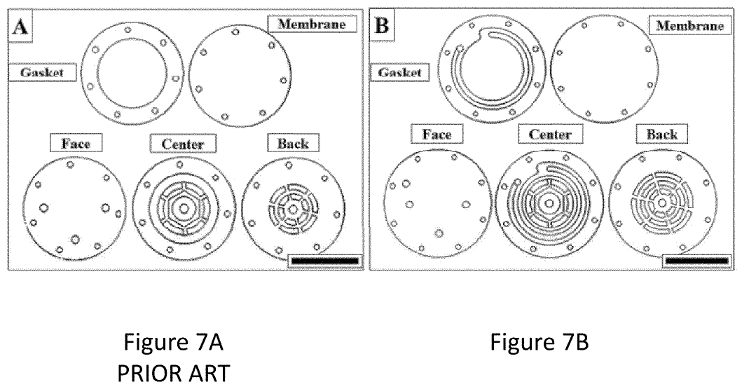

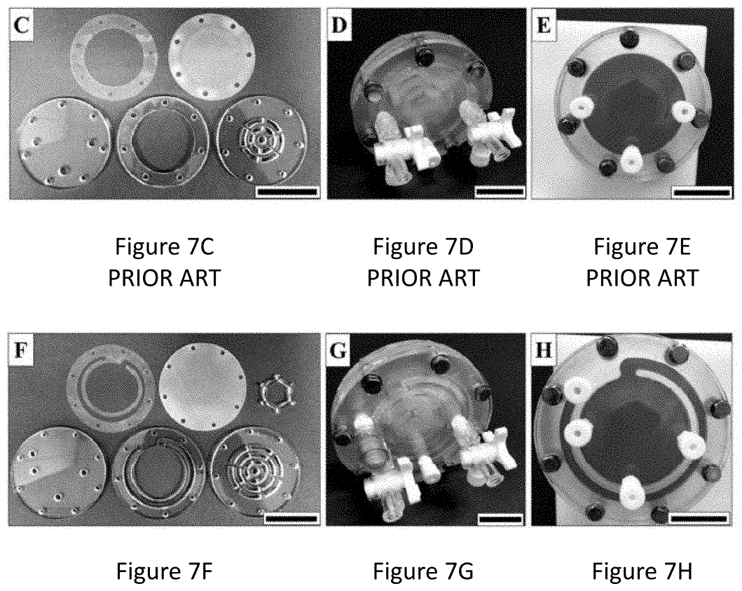

Potential bubble segregating designs were drawn in 2D in AutoCAD 2018 (Autodesk, Mill Valley, Calif.) CAD software. The final design opted for the main body similar to existing RWV bioreactors with an additional thin, exterior, concentric channel running along approximately 80% of the total perimeter, connected to the main body by a thin entrance (FIG. 7). Conceptually, this design relied on buoyancy to continuously direct air bubbles out of the main volume into the channel. The volumes and dimensions of the main body were based off existing disposable 10 ml High Aspect Ratio Vessel (HARV) models (Synthecon, Inc., Houston, Tex.), while the channel was given a volume of approximately 3 ml.

Computational Fluid Dynamics (CFD) was employed to numerically evaluate the fluid dynamics of the new design. The “Fluent” software package in the ANSYS 19.1 release (Ansys, Inc., Canonsburg, Pa.) was used to model fluid behavior in both the novel design and a design emulating the existing 10 ml HARV model in a 2D environment. The simulations were run at 1 G, in water, and at a rotational speed of 10 rpm. The simulations provided the velocity vectors of the fluid at multiple points.

Bioreactor Fabrication:

The bubble capturing bioreactors were designed to be leak-proof, noncytotoxic, sterilizable, and gas permeable for cellular respiration/pH control. Additionally, separate “control” RWVs using identical materials were constructed emulating existing HARV-type bioreactors. The two designs are referred to as “novel” and “standard”, respectively (FIGS. 7A and 7B).

All systems were primarily constructed from 3/16″ cast acrylic ACRYLITE® (Evonik Industries, Essen, Germany) and cut on a VLS 6.60 laser cutting table (Universal Laser Systems, Scottsdale, Ariz.). There were three primary pieces for each bioreactor (FIGS. 7C and 7F). First, a front plate served to close the system from the front and to provide holes for the addition of filling and emptying ports by tapping ¼″-28 holes and connecting to screw-to-Luer Lock Quick Turn Couplings (McMaster-Carr, Elmhurst, Ill.). The front plates of the novel bioreactors contained an additional port at the terminal end of the channel fitted with a pressure activated Luer valve for filling and bubble removal. Next, a central acrylic ring served as the perimeter wall for the fluid with the novel variants containing the bubble catch channel. Finally, the back wall served as a vented backing for gas perfusion and as an anchor point for attachment to a rotation system via a ⅝″-11 nylon nut (McMaster-Carr). All acrylic pieces were pre-cut to accept M4 bolt holes around their perimeter for assembly. After taping to add thread, all acrylic pieces were annealed in an 85° C. oven which had its temperature lowered 10° C. every hour until room temperature was reached. A gas-permeable membrane was produced by laminating a sheet of Tegaderm (3M, Maplewood, Minn.), a known watertight, gas-permeable polymer, onto a flexible nylon mesh with a 300 μm pore size (McMaster-Carr) and tightly compressing under a vice overnight to ensure bonding (Yu-shuang & Jiong, 2009). The nylon-mesh-backed Tegaderm® was then laser-cut to match the size and bolt holes of the acrylic pieces. Finally, 0.5 mm-thick heat-resistant silicone sheet (Laimeisi Silicone, Ltd., Shenzhen, China) was laser cut to match the shape of the central acrylic ring to act as compressible leak-resistant gaskets. Finally, the pieces were assembled in the following order: front acrylic plate, silicone gasket, central acrylic ring, silicone gasket, gas-permeable membrane (Tegaderm facing central ring), back acrylic plate, nylon nut. The assembled bioreactor was then held together with M4×16 bolts with two flat washers, one spring washer, and one bolt each. Stopcocks and Luer closures (McMaster-Carr) were added to enable filling (FIGS. 7D and 7G).

Alginate Bead Fluid Dynamics Validation:

To validate the predicted motion of spheroids in the bioreactors, calcium alginate beads (approximately 2 mm in diameter) were used that were produced by dropwise adding a mixture 0.75% sodium alginate (Sigma-Aldrich, St. Louis, Mo.), 1% polyethylene oxide (Sigma-Aldrich, St. Louis, Mo.), and 2% powdered charcoal (for color) into a stirred 2% calcium chloride bath. Approximately two dozen beads were loaded into both types of bioreactors with and without added air bubbles to replicate ideal and failure conditions and rotated at 25 rpm until each system had achieved stable fluid dynamics, as inferred from reduced motion relative to any fixed position on the device. Motion and velocities of beads at the exterior perimeter of the systems were tracked, essentially as previously described (Botta, Manley, Miller, & Lelkes, 2006), using tripod-mounted GoPro camera (GoPro, Inc., San Mateo, Calif.) at a rate of 60 frames per second. The captured images were subsequently evaluated using ImageJ Manual Tracking relative to bubbles and fixed locations on the bioreactor over the course of 1 second. Shear in the absence of bubbles was calculated according to a previously published formula (Ramirez, Lim, Coimbra, & Kobayashi, 2003), describing fluid shear on small particles in an RWV bioreactor, as shown in Equation 1,

τ=μ*|V_{F}−V_{P}|/a Equation 1

Where τ is fluid shear stress, μ is the dynamic viscosity of water a 25° C., V_{F} is the calculated fluid velocity of water at the particle's radius if the fluid were acting as a solid body, V_{P} is the observed velocity of the particle, and a is the radius of the particle (Ramirez, Lim, Coimbra, & Kobayashi, 2003). Because no theoretical velocity can be assumed at the bubble, the traditional fluid shear formula, Equation 2, was used when a particle interacted with a bubble,

τ=μ*δx/δy Equation 2

where δχ is the change in distance (velocity) and δγ is the difference in location (Ramirez et al., 2003). Thus, δχ/δγ is the velocity gradient developed in the fluid between the wall and the particle.

Equations 1 and 2 are substantial simplifications of actual shear equations which are presented as a complex 3D Navier-Stokes equation, the governing equation of motion in fluids (Begley & Kleis, 2000; Munson, Okiishi, Huebsch, & Rothmayer, 2012; Wolf & Schwarz, 1991). All shear values determined from the above equations are reported in dynes/cm2.

Cell Culture for Morphological Assessment:

To perform cell culture, a new sterilization and conditioning protocol was developed and tested. Since the selected materials are not autoclavable and are prone to degradation upon sterilization by 70% ethanol, the protocol was modified from an existing HARV protocol (Botta, Manley, Miller, & Lelkes, 2006). In brief, each bioreactor was filled completely with 0.2 N NaOH for 24 hours. All bioreactors were then moved to a laminar flow hood and exposed to UV sterilization for 30 minutes on each side. They were then emptied of NaOH and rinsed three times with sterile 1×PBS (Corning, Corning, N.Y.). Next, the bioreactors were filled with a sterile conditioning solution composed of 10% FBS (Gibco, Gaithersburg, Md.), 88% lx PBS (Corning, Corning, N.Y.), and 2% Penicillin Streptomycin (Pen-Strep, Gibco, Gaithersburg, Md.) and incubated at 37° C. for 24 hours while rotating at 10 rpm on a 4RCCS rotary system (Synthecon, Inc., Houston, Tex.). After conditioning, bioreactors were emptied aseptically and immediately used for cell culture.

To assess the morphology of the cell-based organoids/spheroids generated in the RWV under ideal conditions, A549 human lung cancer cells (ATCC, Manassas, Va.) were paradigmatically used that have been described extensively in tumor spheroid publications (Maruhashi et al., 2018; Sambale et al., 2015; Zuchowska, Jastrzebska, Chudy, Dybko, & Brzozka, 2017). The cells were maintained in a T75 flask (Falcon) in DMEM (Gibco) supplemented with 10% FBS, 1% Pen-Strep, and 1% L-glutamine (Gibco). Once the cultures reached ˜80-90% confluence, cells were rinsed with PBS and detached using 1× Trypsin (Gibco) and resuspended in DMEM at a concentration of approximately 1×106 cells/ml. Three ml of complete DMEM were added to each bioreactor, followed by 1 ml of the cell suspension. The systems were then completely filled with media using 20 ml syringes (BD, Franklin Lakes, N.J.) and carefully checked to ensure no bubbles were present. For the novel design, the filling was performed through the valve at the terminal end of the channel. Once cells were added, care was taken to ensure the entrance to the channel was oriented vertically to prevent cells incidentally entering the channel. Each bioreactor was then mounted on the 4RCCS system (Synthecon) that was operated at 10 rpm inside a CO₂ incubator maintained at 370 C. After 48 hours, the main volume of each bioreactor was collected into 15 ml conical centrifuge tubes (Corning, Corning, N.Y.). Cells and aggregates were sedimented over 5 minutes and then collected with 1000 μL pipettes. Any standard design bioreactor, which exhibited bubble formation, was excluded from these results so that the impact of bubble formation could be tested under more controlled conditions.

In a separate set of experiments, air bubbles were intentionally introduced to both the novel and standard bioreactors prior to culture, in order to evaluate the effects of bubble formation on A549 aggregate formation and spheroid morphology. The above cell culture steps were followed precisely including the complete filling of the bioreactors with media and the elimination of air bubbles. Subsequently, an air-filled 1 ml syringe (BD) was attached to one stopcock while an empty 1 ml syringe was attached to the other. Approximately 250 μL of air was added to each bioreactor to simulate a moderate to severe bubble failure. The bioreactors were then operated under precisely the same conditions as the ideal group. The two groups will be referred to as “without bubble” and “with bubble”, respectively.

Imaging and Evaluation:

For both the “with bubble” and “without bubble” groups, the morphology of the ensuing organoids/spheroids was evaluated by standard image analysis of phasecontrast micrographs. At the end of each culture, the contents of each bioreactor were collected, as described above. Using this method, virtually all aggregates and single cells were collected, which was confirmed by visualizing the supernatant. After sedimentation, 300 μL of spheroid-containing media was collected from the bottom of the tubes and gently transferred to the 14 mm centers of 35 mm glass bottom Petri dishes (MatTek, Ashland, Mass.). At least 10 random images per aliquot were taken of all available spheroids, aggregates, or masses of individual cells at 40× magnification. Images were exported to ImageJ, all identifiable spheroids were manually outlined, and data recorded on each image for area and circularity.

Novel Design Failure Mode Analysis:

One possible limitation of this device is the probability of cells entering the channel prior to the establishment of solid body rotation. To evaluate the probability of this occurrence, Human Dermal Fibroblasts (HDFBs from ATCC) were cultured in complete DMEM (Gibco), as described previously (Lin et al., 2012). One hundred thousand HDFBs were added to each of the bioreactors and incubated on the 4RCCS at 10 rpm for 24 hours. HDFBs were selected due to their tendency not to form aggregates, reducing error due to changing sedimentation rates. After 24 hours, the media in the main volume and the channel volume were collected into separate conical tubes through the main and channel ports respectively, spun down, and resuspended in 1 ml of media. Cell counting was then performed three times on each sample and averages were taken of the totals.

Statistics:

Unless stated otherwise, a minimum of three independent experiments (n=3) were performed for each parameter tested. The calculated areas of the organoids/spheroids are presented as medians±the standard deviation of the medians. Circularity and cell count values are shown as averages±the standard deviation of the medians. Significance was evaluated with a two-tailed student t-test and results were considered significant with a p-value less than 0.05.

Results

Design, Modeling, and Computational Fluid Dynamics:

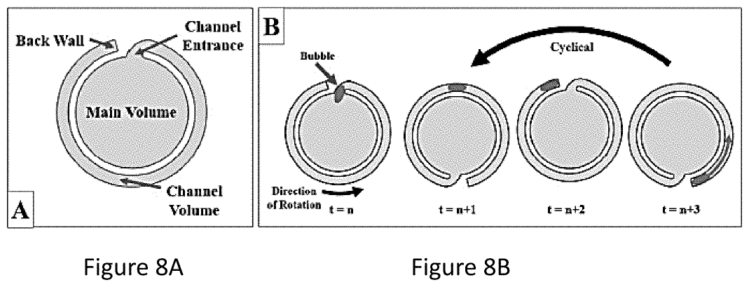

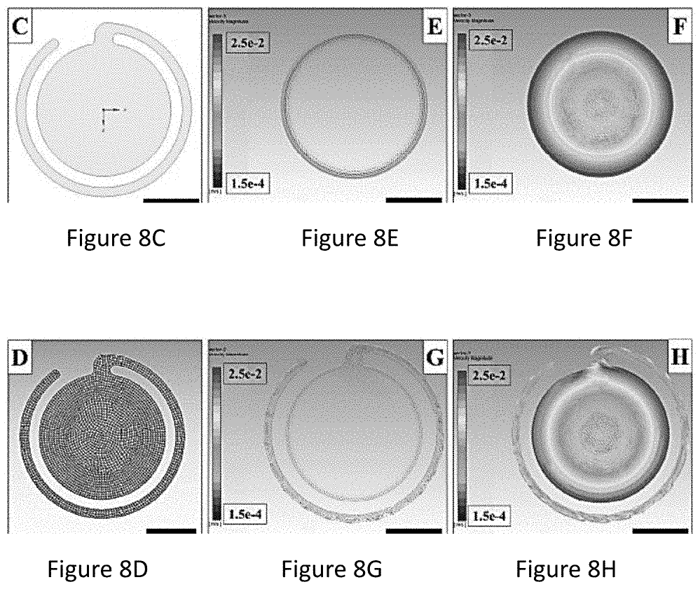

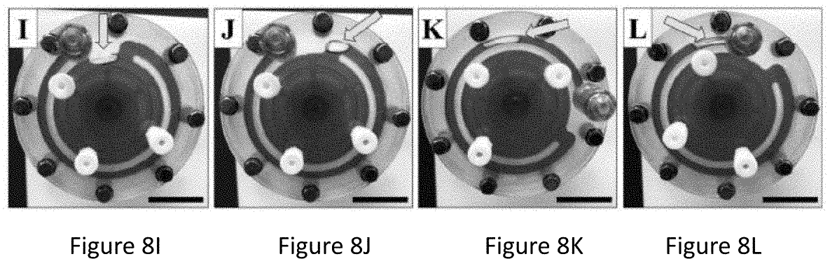

FIGS. 8A-8L shows the design of the novel bioreactor and the results of a computational fluid dynamics analysis comparing the standard and novel designs under ideal conditions, as described above. Shown in FIG. 8A is the concept of the interior volume, comprising the main volume and the volume of the peripheral channel. FIG. 8B demonstrates the general theory of the design, which ensures that due to either buoyancy or sedimentation any small object entering the bubble-capturing channel will unlikely reenter the main volume of the main chamber as defined in FIG. 8A. FIGS. 8C and 8D show the 2D CAD drawing used to model these dynamics and the distribution and size of voxels throughout the simulated system. FIGS. 8E, 8F-8H compare the theoretical fluid dynamics of the standard and novel designs after one second and fifteen minutes of rotation, respectively. While the flow velocities in the main chambers of FIGS. 8F and 8H are strikingly similar, some difference was found around the extreme perimeters of the modeled bioreactors and at the entrance to the channel, with a maximum difference in similar locations at 0.025 m/s in FIG. 8F and 0.02 m/s in FIG. 8H. However, for the majority of the perimeter, flow velocity was ˜0.025 m/s for both systems. More importantly, the ideal operative range of where a particle would be suspended in the system is not the extreme perimeter. Rather, in an ideally functioning bioreactor, particles would need to be several millimeters away from the extreme boundary to prevent wall collisions (Liu, Li, Sun, Ma, & Cui, 2004). Outside of the extreme perimeters of the bioreactors, the fluid velocities of the two systems match nearly identically, approximately 0.01 m/s, and both exhibit highly circular morphology. FIGS. 8I and 8J demonstrate in an assembled device how a bubble is captured through the channel entrance and FIGS. 8K and 8L show the bubbles' path towards the back wall of the channel as the bioreactor rotates.

Bioreactor Fabrication:

Multiple copies of both the standard and novel designs were fabricated; they were leak-proof and functional according to the performance parameters of existing 10 ml HARVtype RWV bioreactors. FIG. 1 shows the finished products of the standard and novel designs and visualizes their internal volumes using dyed water with stopcocks replaced by simple caps for increased visibility. The standard and novel HARVs have measured volumes of approximately 10 ml and 12.5 ml, respectively, with the novel channel representing 25% that of the main volume. The interior dimensions of these vessels are shown in FIG. 7E, 7H.

Alginate Bead Fluid Dynamics Validation:

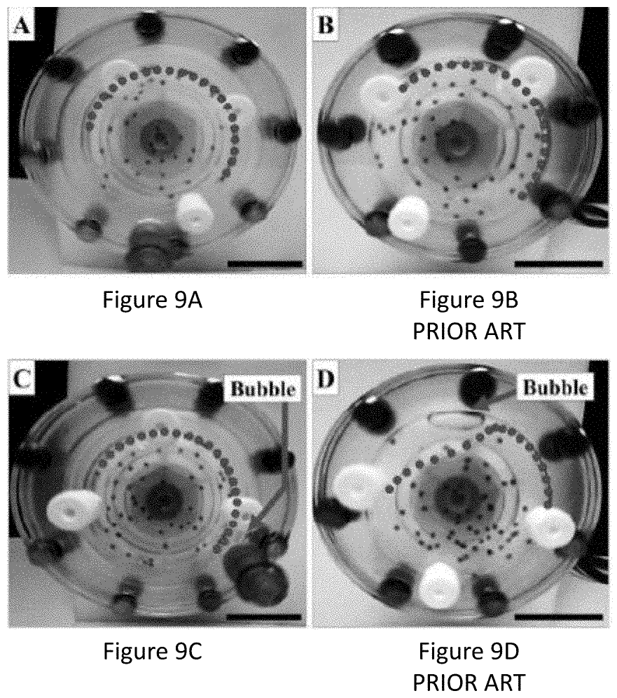

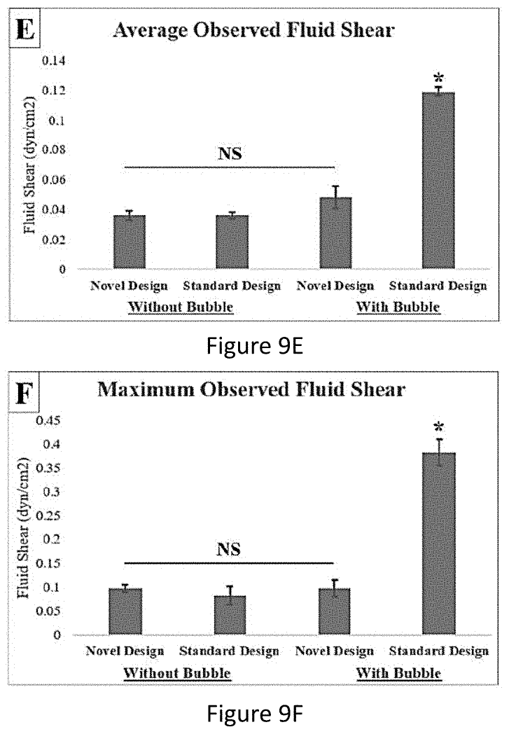

FIGS. 9A-9D show the change in the position of a particular alginate bead over one second of rotation of the novel bioreactors (FIGS. 9A, 9C) and the standard bioreactors (FIGS. 9B, 9D) with and without bubbles after each system had reached equilibrium, with the red dots tracking the motion of one particle every 1/20th of a second (FIGS. 9A-9D) and the subsequently calculated shear values from Equation 1 (FIGS. 9E, 9F). Notably, the deflection of the path of the bead in FIG. 9D (standard bioreactor with bubble) indicates the fluid dynamic disruption in the RWV by a single bubble. Though initially the path appears similar to the other three conditions, as soon as the particle interacts with the bubble, it slows, is deflected off of its course, and then is accelerated into a new position. This altered movement destroys the whole-body rotation (zero headspace, simulated microgravity) condition due to the disruption of the circular path required to establish the clinostat effect, while also increasing the shear forces as much as five-fold (FIG. 9F). The particle paths in FIGS. 9A-9C look remarkably similar, indicating that the bubble-capturing bioreactor maintains the ideal environment zero headspace/whole body rotation by effectively removing the intentionally introduced bubble. FIGS. 9E and 9F demonstrate the insignificant differences in both average and maximum shear experienced in the above three conditions. Although a bubble is not readily visible in FIG. 9C, it is present at the back of the channel (red arrow) and is of the same size as that in FIG. 9D.

Cell Culture:

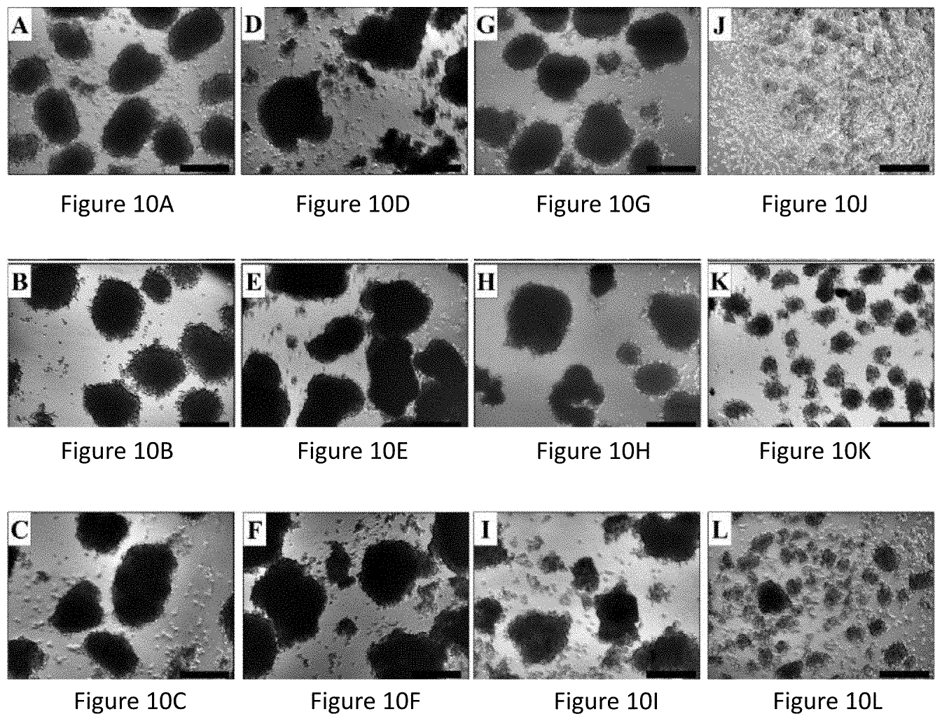

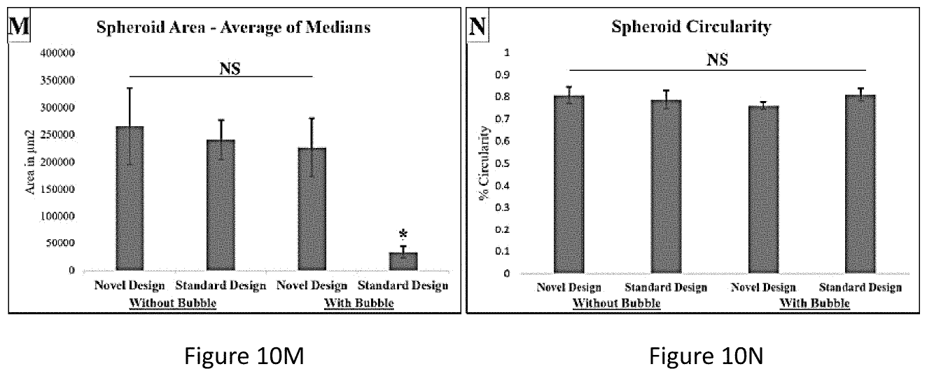

Shown in FIGS. 10A-10L are micrographs of three independent A549 spheroid cultures from each of the four conditions described above. FIGS. 10A-10C show typical spheroids from the novel bioreactor without air bubbles, FIGS. 10D-10F from the standard bioreactor without air bubbles, FIGS. 10G-10I from the novel bioreactor with an added air bubble, and FIGS. 10J-10L from the standard bioreactor with an added air bubble. The images in FIGS. 10A-10I shows strikingly similar results in terms of spheroid size and circularity. By contrast, only either small aggregates or single cell suspensions were found in the standard bioreactor in the presence of bubbles (FIGS. 10J-10L). This difference is outlined in FIG. 10M, showing an approximately fivefold difference in area between the first three groups and the last. Interestingly, the circularity of the spheroids seems unaffected between the four groups regardless of condition or spheroid size.

Of note, the volumes of the channels were also collected from the novel design bioreactors. While there were generally little to no aggregates or single cells present, on occasion, a few very large spheroids (of sufficient scale to fall out of suspension, typically >500 μm at the chosen rotational speeds) were found. This may indicate a tendency for spheroids to irreversibly enter the channel, once their sedimentation rates exceed the maximum velocity of the rotating media.

Novel Design Failure Mode Analysis:

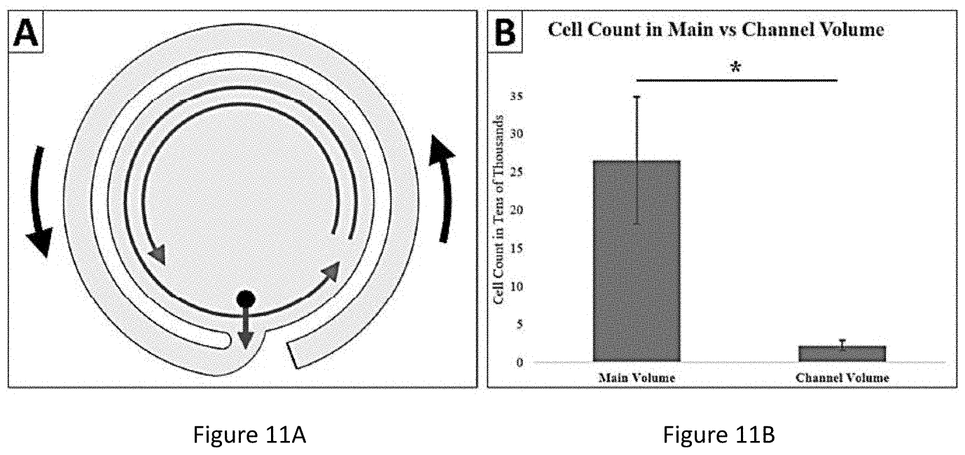

FIGS. 11A and 11B describe the analysis of the potential failure mode of the novel bioreactor, i.e. of cells irreversibly falling into the novel channel over time (FIG. 11A). To assess this possibility, Human Dermal Fibroblasts were seeded into the main volume and cultured for 24 hours, before harvesting and counting the cells to determine the totals in the main and channel volumes. Although there is a finite probability for cells to enter the channel during culture, the total number of cells found in the channel over a 24-hour period was less than 2%, as shown in FIG. 11B.

Discussion

The aim of this study was to design a modified RWV capable of producing the same low-shear, microgravity-simulating environment as a typical RWV, while capturing and removing nascent bubbles, thus maintaining the critical zero headspace condition. The results in FIGS. 9A-10N demonstrate that the modified RWV can produce and maintain the required conditions stated above, while actively removing bubbles and producing spheroids like traditional RWV devices under optimal, bubble-free conditions.

While the equations used to calculate shear in the alginate bead systems are substantial simplifications, the calculated values, an average ˜0.04 dynes/cm2, under ideal conditions very closely approximated the previously published ideal range of −0.044 dynes/cm2 under zero headspace conditions (Begley & Kleis, 2000). By contrast, in the presence of a bubble (FIG. 3D), the fluid shear stress increases approximately 10-fold to 0.4 dynes/cm2. This order of magnitude increase would be sufficient to disrupt aggregate formation, but may not induce cell apoptosis, which reportedly requires 0.92 dynes/cm2 (Goodwin, Prewett, Wolf, & Spaulding, 1993). When taken contextually with FIGS. 10J-10L, the appearance of single cells and very small aggregates (an order of magnitude smaller than the other three groups) indicates that aggregate formation was indeed disrupted as a result of increased shear forces. This observation serves not only to strengthen the assumptions made in the calculations of the fluid shear in the main volume of the bioreactors but also to provide an explanation of the observed cell culture results. Juxtaposed with the loss of aggregates in the standard bioreactor design with bubble, the insignificant differences (FIGS. 10M, 10N) in aggregate size in the novel design with bubbles as compared to the no bubble conditions indicates that this novel technology achieved its intended goal of reducing the impact of bubbles on aggregate formation. Our results demonstrate that this device may combine enhanced reproducibility of organoid generation with improved ease of use and a decreased likelihood of experiment failure. Though the probability of “channel capture” may increase as spheroids grow and sedimentation rates change, this issue can be resolved by increasing the rotational speed of the device.

The prevention of bubble formation is important for the outcome and reproducibility of the results, yet seemingly underreported in the literature (T. G. Hammond & Hammond, 2001). Because bubble formation is frequent, and its prevention/removal is anecdotal, at best, researchers are likely to collect results from RWVs that intermittently formed bubbles (e.g., overnight), not realizing it actually constitutes a flawed experiment. As the use of RWV bioreactor systems is expanding, the presence of bubbles may yield data on unique cellular behavior in these vessels that may, in fact, represent the opposite of what would occur in actual low-shear microenvironement. It is one of our intentions that the dissemination of the novel design may be able to prevent false positive/negative results and increase the reproducibility of the system.

Although the calculated shear stresses and the observed cell spheroid sizes were statistically indistinguishable between the “standard without bubble” group and either of the “novel” groups, there are differences between the standard and novel designs. As described in FIG. 5, there is a probability of a small subpopulation of the cells irreversibly entering the novel channel. FIG. 5B shows this probability is low (less than 2% over 24 hours) if the rotation speed of the system is in the correct range. However, channel capture is only likely to occur if the cells or aggregates tend to drift towards the exterior perimeter of the main volume, with the probability of that tendency increasing if the rotational velocity is incorrect. In the standard system, a device set to the incorrect speed would still contain all its cell contents. In the novel system, if an incorrect rotational speed is set, aggregates would tend to be captured, thus providing tangible feedback that the system settings are inadequate.

Furthermore, in a standard system, any particles interacting with the wall may lose their circular path, negating the modeled microgravity (whole body rotation, zero headspace) environment. However, these cells would still be collected together with the entire bioreactor contents, potentially skewing mechanistic analyses, such as global gene expression profiling in the wake of organoid assembly and differentiation. In the novel system, cells in the main volume would be collected separately from those in the channel volume, ensuring the cells tested are only the ones that did not tend towards the wall, decreasing skew and increasing the consistency of results. Finally, because aggregates grow over time, the tendency for larger aggregates to enter the channel may be leveraged as a mechanism to harvest aggregates of uniform sizes with potential applications in industrial GMP settings.

In summary, a modification to the traditional HARV-type rotating wall vessel bioreactor was engineered and tested, which results in the effective capture and removal of bubbles. The modified and standard bioreactors were compared side by side and found similarities in both fluid dynamics and in the ability to generate cancer spheroids in the absence of bubbles. However, in the presence of a bubble, the spheroid/organoid formation is impaired in conventional RWVs, while it is fully maintained in the modified design. The novel design will increase experimental reproducibility and consistency when using rotating wall vessel bioreactors.

### REFERENCES

Antoni, D., Burckel, H., Josset, E., & Noel, G. (2015). Three-dimensional cell culture: a breakthrough in vivo. International Journal of Molecular Sciences, 16, 5517-5527.
Begley, C. M., & Kleis, S. J. (2000). The Fluid Dynamic and Shear Environment in the NASA/JSC Rotating—Wall Perfused-Vessel Bioreactor. Biotechnol Bioeng, 70, 32-40.
Botta, G. P., Manley, P., Miller, S., & Lelkes, P. I. (2006). Real-time assessment of three-dimensional cell aggregation in rotating wall vessel bioreactors in vitro. Nature Protocols, 1, 2116-2127.
DiStefano, T., Chen, H. Y., Panebianco, C., Kaya, K. D., et al. (2018). Accelerated and Improved Differentiation of Retinal Organoids from Pluripotent Stem Cells in Rotating-Wall Vessel Bioreactors. Stem Cell Reports, 10, 300-313. Eder, T., & Eder, I. E. (2017). 3D Cell Culture Methods and Protocols (pp. 167-175). Humana Press, New York, N.Y.
Fang, Y., & Eglen, R. M. (2017). Three-Dimensional Cell Cultures in Drug Discovery and Development. SLAS Discovery: Advancing Life Sciences R & D, 22, 456-472.
Gerecht-Nir, S., Cohen, S., & Itskovitz-Eldor, J. (2004). Bioreactor Cultivation Enhances the Efficiency of Human Embryoid Body (hEB) Formation and Differentiation. Biotechnol Bioeng, 86, 493-502.
Goodwin, T. J., Prewett, T. L., Wolf, D. A., & Spaulding, G. F. (1993). Reduced shear stress: A major component in the ability of mammalian tissues to form three-dimensional assemblies in simulated microgravity. Journal of Cellular Biochemistry, 51, 301-311.
Hammond, T. G., Allen, P., & Birdsall, H. (2016). Is There a Space-Based Technology Solution to Problems with Preclinical Drug Toxicity Testing? Pharmaceutical Research, 33, 1545-1551.
Hammond, T. G., & Hammond, J. M. (2001). Optimized suspension culture: the rotating-wall vessel. Am J Physiol Renal Physiol, 281, F12-25.
King, J. A., & Miller, W. M. (2007). Bioreactor Development for Stem Cell Expansion and Controlled Differentiation. Current Opinion in Chemical Biology, 11, 394-398.
Lelkes, P. I., Galvan, D. L., Thomas Hayman, G., Goodwin, et al. (1998). Simulated microgravity conditions enhance differentiation of cultured PC12 cells towards the neuroendocrine phenotype. In Vitro Cellular and Developmental Biology—Animal, 34, 316-325.
Lin, L., Perets, A., Har-El, Y.-E., Varma, D., Li, M., et al. (2012). Alimentary “green” proteins as electrospun scaffolds for skin regenerative engineering. J Tissue Eng Regen Med, 7, 994-1008.
Liu, T., Li, X., Sun, X., Ma, X., & Cui, Z. (2004). Analysis on forces and movement of cultivated particles in a rotating wall vessel bioreactor. Biochemical Engineering Journal, 18, 97-104.
Maruhashi, R., Akizuki, R., Sato, T., Matsunaga, T., et al. (2018). Elevation of sensitivity to anticancer agents of human lung adenocarcinoma A549 cells by knockdown of claudin-2 expression in monolayer and spheroid culture models. Biochimica et Biophysica Acta (BBA)—Molecular Cell Research, 1865, 470-479.
Mattei, C., Alshawaf, A., D 'abaco, G., Nayagam, B., & Dottori, M. (2018). Generation of Neural Organoids from Human Embryonic Stem Cells Using the Rotary Cell Culture System: Effects of Microgravity on Neural Progenitor Cell Fate. Stem Cells and Development, 27, 848-857.
Munson, Okiishi, Huebsch, & Rothmayer. (2012). Fluid Mechanics (7th ed.). John Wiley & Sons, Inc., Hoboken, N.J.
Pollack, S. R., Meaney, D. F., Levine, E. M., Litt, M., & Johnston, E. D. (2000). Numerical Model and Experimental Validation of Microcarrier Motion in a Rotating Bioreactor. TISSUE ENGINEERING, 6, 519-530.
Radtke, A. L., Herbst-Kralovetz, M. M. Culturing and Applications of Rotating Wall Vessel Bioreactor Derived 3D Epithelial Cell Models. J. Vis. Exp. (62), e3868. doi:10.3791/3868 (2012).
Ramirez, L. E. S., Lim, E. A., Coimbra, C. F. M., & Kobayashi, M. H. (2003). On the dynamics of a spherical scaffold in rotating bioreactors. Biotechnology and Bioengineering, 84, 382-389.
Redden, R. A., & Doolin, E. J. (2011). Microgravity assay of neuroblastoma: in vitro aggregation kinetics and organoid morphology correlate with MYCN expression. In Vitro Cell Dev Biol Anim, 47, 312-317.
Redden, R. A., Iyer, R., Brodeur, G. M., & Doolin, E. J. (2014). Rotary bioreactor culture can discern specific behavior phenotypes in Trk-null and Trk-expressing neuroblastoma cell lines. In Vitro Cell Dev. Biol, 50, 188-193.
Salerno-Goncalves, R., Fasano, A., & Sztein, M. B. (2016). Development of a Multicellular Threedimensional Organotypic Model of the Human Intestinal Mucosa Grown Under Microgravity. J. Vis. Exp., (113).
Sambale, F., Lavrentieva, A., Stahl, F., Blume, C., et al. (2015). Three dimensional spheroid cell culture for nanoparticle safety testing. Journal of Biotechnology, 205, 120-129.
Schwarz, R. P., Goodwin, T. J., & Wolf, D. A. (1992). Cell culture for three-dimensional modeling in rotating-wall vessels: An application of simulated microgravity. Journal of Tissue Culture Methods, 14, 51-57.
Varley, M. C., Markaki, A. E., & Brooks, R. A. (2017). Effect of Rotation on Scaffold Motion and Cell Growth in Rotating Bioreactors. Tissue Engineering. Part A, 23, 522-534.
Wolf, D. A., & Schwarz, R. P. (1991). Analysis of gravity-induced particle motion and fluid perfusion flow in the NASA-designed rotating zero-head-space tissue culture vessel [NASA Technical Paper].
Wolf, D. A., & Schwarz, R. P. (1992). Experimental measurement of the orbital paths of particles sedimenting within a rotating viscous fluid as influenced by gravity [NASA Technical Paper].
Wuest, S. L., Richard, S., Kopp, S., Grimm, D., & Egli, M. (2015). Simulated microgravity: critical review on the use of random positioning machines for mammalian cell culture. BioMed Research International, 2015.
Xinaris, C., Brizi, V., & Remuzzi, G. (2015). Organoid Models and Applications in Biomedical Research. Nephron, 130, 191-199.
Yu-shuang, L., & Jiong, C. (2009). Moisture vapor transmission rates of various transparent dressings at different temperatures and humidities. Chinese Medical Journal, 122, 927-930.
Zuchowska, A., Jastrzebska, E., Chudy, M., Dybko, A., & Brzozka, Z. (2017). 3D lung spheroid cultures for evaluation of photodynamic therapy (PDT) procedures in microfluidic Lab-on-a-Chip system. Analytica Chimica Acta, 990, 110-120.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A culture chamber for a rotary bioreactor comprising:
a central chamber;
an elongate channel comprising a channel entrance configured to receive bubbles from the central chamber;
wherein the elongate channel surrounds a majority of the central chamber.

2. The culture chamber of claim 1, wherein the channel entrance comprises a back wall that is common to an outer wall to the elongate channel.

3. The culture chamber of claim 1, wherein the elongate channel terminates in a channel exit port.

4. The culture chamber of claim 1, wherein the elongate channel is curved.

5. The culture chamber of claim 1, wherein the curvature of the elongate channel approximates a curvature of the perimeter of the central chamber.

6. The culture chamber of claim 1, wherein the elongate channel surrounds the entire central chamber.

7. The culture chamber of claim 1, wherein the elongate channel extends beyond one complete turn around the central chamber.

8. The culture chamber of claim 1, wherein the channel entrance comprises a gating mechanism.

9. The culture chamber of claim 8, wherein the gating mechanism is one of a hydrophobic membrane, a ball valve or a one-way flow valve.

10. The culture chamber of claim 1, wherein the culture chamber is substantially disc-shaped.

11. The culture chamber of claim 1, wherein the culture chamber is substantially drum-shaped.

12. The culture chamber of claim 11, wherein the channel entrance is disposed at the vertex of at least one angled ramp.

13. The culture chamber of claim 11, wherein the channel entrance is disposed between and at the vertex of at least two angled ramps.

14. The culture chamber of claim 11 further comprising:
an outer sheath at least partially comprising the elongate channel.

15. A culture chamber assembly comprising a plurality of disks that form the culture chamber of claim 1.

16. The culture chamber assembly of claim 15 comprising:
a gas-permeable membrane layer.

17. The culture chamber assembly of claim 15 comprising:
a gasket layer at least partially comprising one of the central chamber and the elongate channel.

18. A bioreactor system comprising:
the culture chamber of claim 1; and
a rotator device configured to attach to and rotate the culture chamber.

19. A HARV or STLV simulated microgravity kit comprising the culture chamber of claim 1.

20. The culture chamber of claim 1, wherein the channel entrance shares the same plane as the central chamber.

21. The culture chamber of claim 1, wherein the channel entrance and the central chamber are configured in different planes.

22. The culture chamber of claim 1, further comprising:
a second elongate channel comprising a second channel entrance configured to receive bubbles from the central chamber.

23. The culture chamber of claim 22, wherein the channel entrance, the second channel entrance and the central chamber are all configured in different planes.

23. The culture chamber of claim 22, wherein the central chamber is configured to accept a working volume of 10 mL of media or less.

24. A method for removing bubbles from a cell culture comprising:
introducing media into a culture chamber;
continuously rotating the culture chamber;
affecting the movement of bubbles from a central chamber of the culture chamber to a peripheral channel of the culture chamber as the culture chamber rotates; and
maintaining the bubbles in the peripheral channel as the culture chamber rotates.

25. The method of claim 24, wherein the step of introducing media into a culture chamber comprises introducing media of 10 mL or less.
